(19) United States
(12) Patent Application Publication
(10) Pub. No.: US 2023/0298083 A1
(43) Pub. Date: Sep. 21, 2023

(54) COMMERCE-BASED SHOPPING EXPERIENCE WITH DIGITAL TWIN SIMULATION INFUSION

(71) Applicant: International Business Machines Corporation, Armonk, NY (US)

(72) Inventors: Christian Compton, Austin, TX (US); Jeremy R. Fox, Georgetown, TX (US); Tanner Mark Preiss, Schnecksville, PA (US); Sarbajit K. Rakshit, Kolkata (IN)

(21) Appl. No.: 17/655,236

(22) Filed: Mar. 17, 2022

(51) Int. Cl. G06Q 30/06; G06F 16/26

(52) U.S. Cl. CPC G06Q 30/0633; G06F 16/26

(57) ABSTRACT

In an approach for creating a digital twin representative of a user and the food consumption of the user and for performing a simulation of the digital twin to create a food buying plan for a shopping cycle, a processor creates a digital twin representative of a user. A processor mines for data about the user from a plurality of sources to personalize the digital twin. A processor performs a simulation of the digital twin to create a food buying plan for a shopping cycle. A processor identifies a price of each food item on the food buying plan and one or more available offers on each food item on the food buying plan to determine whether a monetary budget for the shopping cycle is sufficient. Responsive to determining the monetary budget for the shopping cycle is sufficient, a processor outputs a shopping list for the shopping cycle.

### BACKGROUND OF THE INVENTION

The present invention relates generally to the field of digital twins, and more particularly to a commerce-based shopping experience with a digital twin simulation infusion.

A digital twin is a virtual representation of a physical object, system, or other asset (hereinafter referred to as a “physical asset”) across its life-cycle. The digital twin integrates Internet of Things (IoT), Artificial Intelligence (AI), machine learning, and software analytics with spatial network graphs to create a complex virtual model that is an exact counterpart to the physical asset existing in real space. The digital twin tracks changes to the physical asset across the physical asset’s life-cycle and records the changes as they occur. Sensors and IoT devices connected to the physical asset collect data, often in real-time. The collected data can then be mapped to the virtual model of the digital twin. Any individual with access to the digital twin can see real-time information about the physical asset while the physical asset is operating in the real world without having to be physically present. The individual with access to the digital twin can use the digital twin to understand not only how the physical asset is performing, but to also predict how the physical asset may perform in the future, using the data collected from sensors, IoT devices, and other sources of data and information being collected.

The digital twin finds an application in many fields but mainly in product lifecycle management, advanced manufacturing, project planning, reliability engineering, training, real-time decision making, and decommissioning resources. For example, the digital twin can ensure that the requirements captured during the earliest stages of a physical asset’s lifecycle are verified, maintained, and completed as the physical asset is built, enters service, evolves during service, and is ultimately retired, decommissioned, and recycled. In another example, the digital twin can compare different lifecycle plans based on an impact from other digital twins as the digital twins evolve to assist with contingency and resilience management to ensure a plan is achievable. In yet another example, the digital twin can monitor performance of a physical asset in as near to real time as is needed and can digitally reflect sensor information from the real-world instance of the physical asset so that engineers can improve overall performance, increase system efficiency, and optimize behavior.

While the digital twin finds an application in the fields previously mentioned, the digital twin has also been adopted in the healthcare industry to improve and to personalize medicine. Today’s medical digital twin represent a new take on digital twins. Today’s medical digital twin is a patient-specific digital twin (i.e., a virtual representation of the patient in the real physical world). The patient-specific digital twin is generated from data gathered from different data sources, including, but not limited to, the patient’s medical records, omics, and wearable devices (i.e., a networked set of biosensors, wearable technology devices, peripherals, smart pill dispensers, smart inhalers, ingestible smart pills, implantable devices (e.g., implantable cardio defibrillators), smart injectors, smartphone applications, and smart speakers). The patient-specific digital twin analyzes the data gathered from the different data sources to uncover information representative of the real person’s own health and lifestyle as well as to provide feedback, such as diagnoses, predictions, and other suggestions.

### SUMMARY

Aspects of an embodiment of the present invention disclose a method, computer program product, and computer system for creating a digital twin representative of a user and the food consumption of the user and for performing a simulation of the digital twin to create a food buying plan for a shopping cycle. A processor creates a digital twin representative of a user in an unmodified state. A processor mines for data associated with the user from a plurality of sources to personalize the digital twin. A processor performs a simulation of the digital twin to create a food buying plan for a shopping cycle. A processor identifies a price of each food item on the food buying plan and one or more available offers on each food item on the food buying plan to determine whether a monetary budget for the shopping cycle is sufficient. Responsive to determining the monetary budget for the shopping cycle is sufficient, a processor outputs a shopping list for the shopping cycle based on the simulation of the digital twin.

In some aspects of an embodiment of the present invention, a processor collects a set of user specific data from the user and from a user profile of the user, wherein the set of user specific data includes biometric identifying data and health data. A processor collects a set of weather data from a weather application programming interface. A processor extracts information from the set of user specific data relating to a health condition of the user.

In some aspects of an embodiment of the present invention, prior to performing the simulation of the digital twin to create the food buying plan for the shopping cycle, a processor enables the user to define the shopping cycle. Responsive to receiving a defined shopping cycle from the user, a processor identifies one or more activities of the user that may occur during the shopping cycle based on one or more historical patterns, a set of biometric identifying data of the user, information extracted from the set of user specific data relating to the health condition of the user, and a calendar of the user. A processor receives the monetary budget for the shopping cycle.

In some aspects of an embodiment of the present invention, a processor updates the digital twin to include the set of user specific data, the set of weather data, the health condition of the user, the shopping cycle, the one or more activities of the user that may occur during the shopping cycle, and the monetary budget for the shopping cycle.

In some aspects of an embodiment of the present invention, a processor collects a list of food items available in a stockpile of the user, an accounting of the food items available in the stockpile of the user, and one or more associated details of the food items available in the stockpile of the user. A processor identifies one or more recipes that can be created using the food items available in the stockpile of the user. A processor collects a list of food items available in a stockpile of a plurality of brick-and-mortar businesses, a plurality of online stores, a plurality of warehouses, and a plurality of supply chains; an accounting of the food items available in the stockpile of the plurality of brick-and-mortar businesses, the plurality of online stores, the plurality of warehouses, and the plurality of supply chains; and one or more associated details about the food items available in the stockpile of the plurality of brick-and-mortar businesses, the plurality of online stores, the plurality of warehouses, and the plurality of supply chains.

In some aspects of an embodiment of the present invention, subsequent to performing the simulation of the digital twin to create the food buying plan for the shopping cycle, a processor creates the food buying plan for the shopping cycle. A processor runs a health simulation on the digital twin to determine how the food items on the food buying plan for the shopping cycle will impact the health condition of the user.

In some aspects of an embodiment of the present invention, responsive to determining the monetary budget for the shopping cycle is insufficient to purchase the food items on the food buying plan for the shopping cycle, a processor notifies the user that the monetary budget for the shopping cycle is insufficient and that the user must increase the monetary budget for the shopping cycle. A processor enables the user to change the monetary budget for the shopping cycle.

These and other features and advantages of the present invention will be described in, or will become apparent to those of ordinary skill in the art in view of, the following detailed description of the example embodiments of the present invention.

### BRIEF DESCRIPTION OF THE DRAWINGS

FIG. 1 is a functional block diagram illustrating a distributed data processing environment, in accordance with an embodiment of the present invention;

FIG. 2 is a block diagram illustrating the components of a server within the distributed data processing environment of FIG. 1, in accordance with an embodiment of the present invention;

FIG. 3 is a block diagram illustrating an exemplary cloud computing environment, in accordance with an embodiment of the present invention;

FIG. 4 is a block diagram illustrating a set of functional abstraction model layers of the exemplary cloud computing environment, in accordance with an embodiment of the present invention; and

FIG. 5 is a flowchart illustrating the operational steps of a shopping support program, on the server within the distributed data processing environment of FIG. 1, in accordance with an embodiment of the present invention.

### DETAILED DESCRIPTION

Embodiments of the present invention recognize that a shopper may create a shopping list before shopping in a store so that the types of items and the quantity of items are properly purchased. Embodiments of the present invention recognize that one issue with creating a shopping list is that the type of items and the quantity of items are based on the shopper’s “guess-estimation” and, based on the shopper’s “guess-estimation,” the shopper will then shop for various items. For example, the shopper will look at what the shopper already has (e.g., in the shopper’s stockpile at home), consider what plans the shopper has during the next shopping cycle (e.g., specific day, week, or month), and then shop accordingly. The shopper may not consider changes in available options during the next shopping cycle.

Therefore, embodiments of the present invention recognize the need for a system and method in which a digital twin computing system simulates a user and any changes associated with the user during a shopping cycle (i.e., a change in the user’s health condition, a change in the user’s choices or plans, or a change in weather conditions) and accordingly creates a food buying plan for the user for the shopping cycle.

Embodiments of the present invention provide a system and method to create a digital twin representative of a user and the food consumption of the user, wherein the digital twin is created based on a set of user specific data, a health condition of the user, a change in the health condition of the user, a shopping cycle, a monetary budget for the shopping cycle, one or more activities of the user that may occur during the shopping cycle, and a set of weather data. Additionally, embodiments of the present invention provide a system and method to perform a simulation of the digital twin to create a food buying plan for the shopping cycle.

Implementation of embodiments of the present invention may take a variety of forms, and exemplary implementation details are discussed subsequently with reference to the Figures.

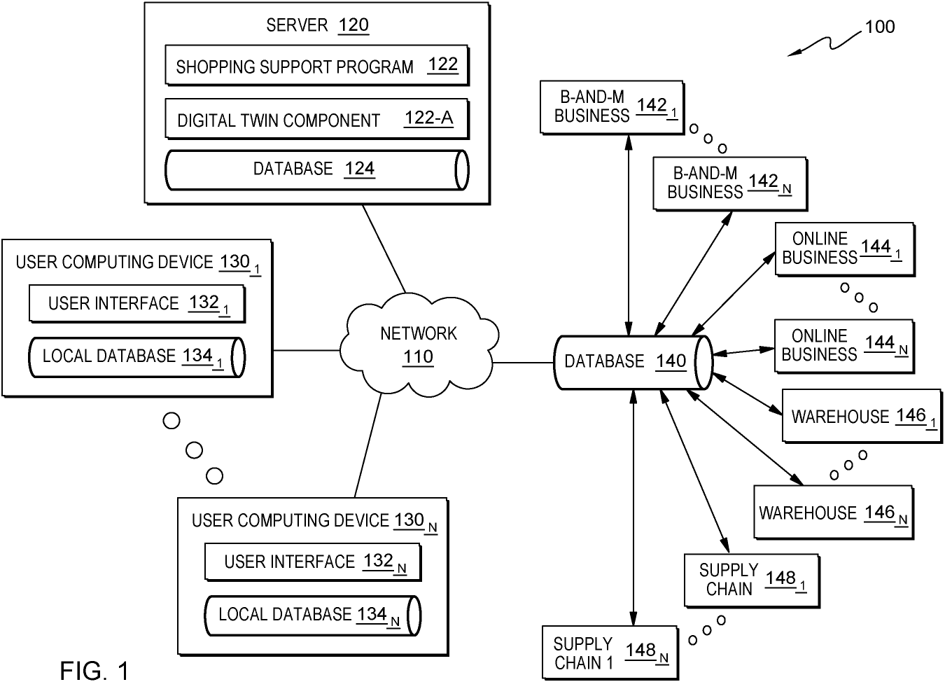

FIG. 1 is a functional block diagram illustrating a distributed data processing environment, generally designated 100, in accordance with an embodiment of the present invention. In the depicted embodiment, distributed data processing environment 100 includes server 120, user computing devices 130_{1-N}, database 140, brick-and-mortar businesses 142_{1-N}, online businesses 144_{1-N}, warehouses 146_{1-N}, and supply chains 148_{1-N}, interconnected over network 110. Distributed data processing environment 100 may include additional servers, computers, computing devices, IoT sensors, and other devices not shown. The term “distributed” as used herein describes a computer system that includes multiple, physically distinct devices that operate together as a single computer system. FIG. 1 provides only an illustration of one embodiment of the present invention and does not imply any limitations with regards to the environments in which different embodiments may be implemented. Many modifications to the depicted environment may be made by those skilled in the art without departing from the scope of the invention as recited by the claims.

Network 110 operates as a computing network that can be, for example, a telecommunications network, a local area network (LAN), a wide area network (WAN), such as the Internet, or a combination of the three, and can include wired, wireless, or fiber optic connections. Network 110 can include one or more wired and/or wireless networks capable of receiving and transmitting data, voice, and/or video signals, including multimedia signals that include data, voice, and video information. In general, network 110 can be any combination of connections and protocols that will support communications between server 120, user computing devices 130_{1-N}, database 140, other computing devices (not shown), and other databases (not shown) within distributed data processing environment 100.

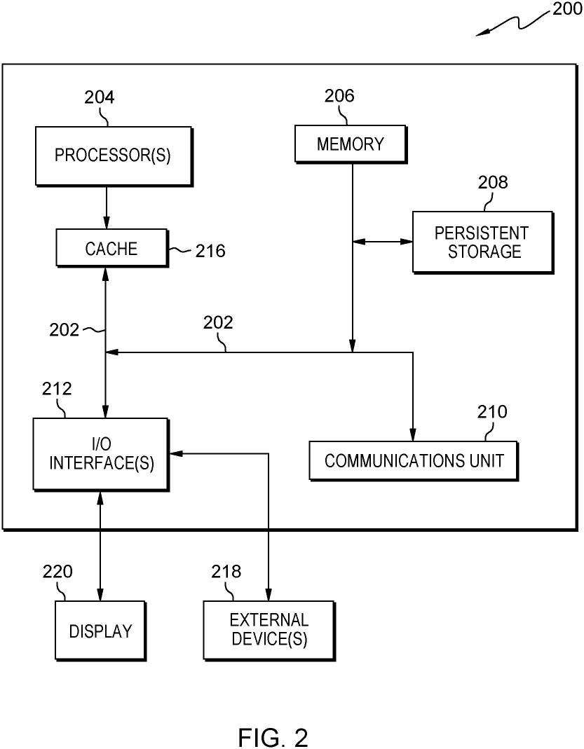

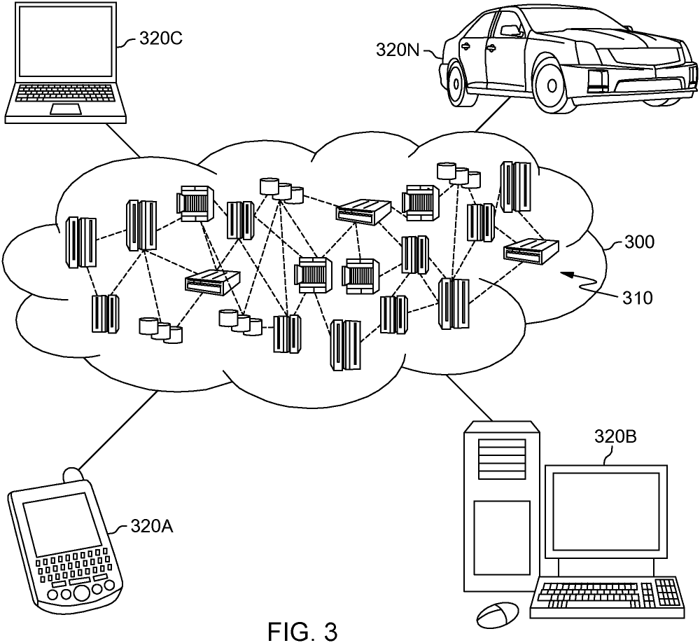

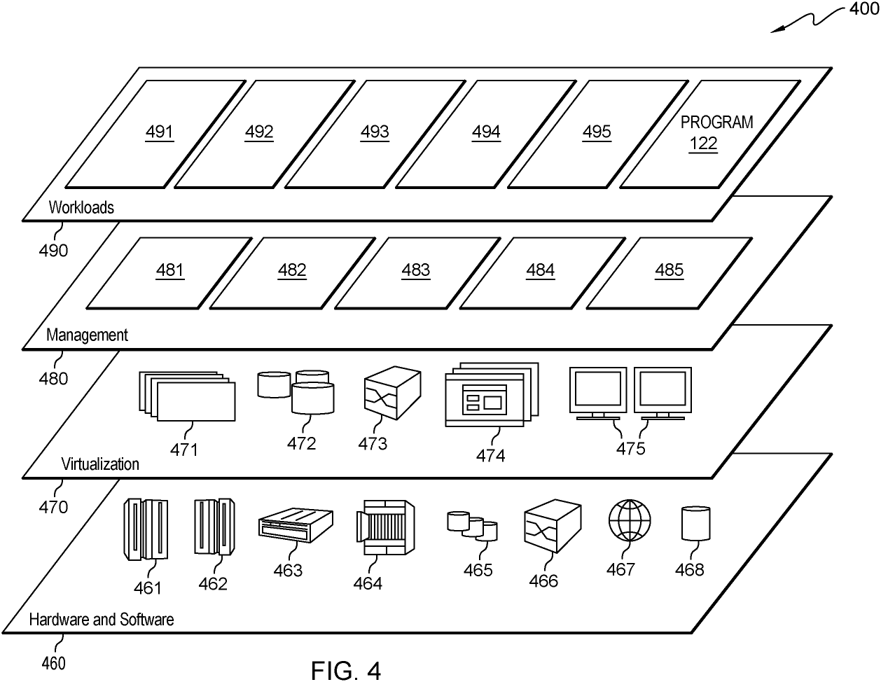

Server 120 operates to run shopping support program 122 and to send and/or store data to database 124. In an embodiment, server 120 can send data from database 124 to user computing devices 130_{1-N}. In another embodiment, server 120 can send data from database 124 to database 140. In an embodiment, server 120 can receive data in database 124 from user computing device 130_{1-N}. In another embodiment, server 120 can receive data in database 124 from database 140. In one or more embodiments, server 120 can be a standalone computing device, a management server, a web server, a mobile computing device, or any other electronic device or computing system capable of receiving, sending, and processing data and capable of communicating with user computing devices 130_{1-N} via network 110. In one or more embodiments, server 120 can be a computing system utilizing clustered computers and components (e.g., database server computers, application server computers, etc.) that act as a single pool of seamless resources when accessed within distributed data processing environment 100, such as in a cloud computing environment, as described in FIGS. 3 and 4. In one or more embodiments, server 120 can be a laptop computer, a tablet computer, a netbook computer, a personal computer, a desktop computer, a personal digital assistant, a smart phone, or any programmable electronic device capable of communicating with user computing devices 130_{1-N}, database 140, other computing devices (not shown), and other databases (not shown) within distributed data processing environment 100 via network 110. Server 120 may include internal and external hardware components, as depicted and described in further detail in FIG. 2.

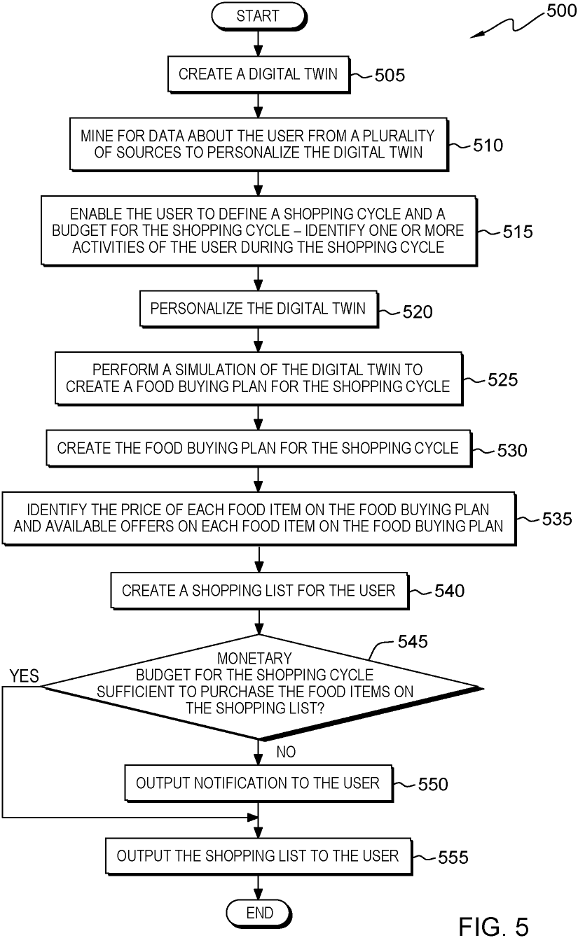

Shopping support program 122 operates to create a digital twin representative of a user and the food consumption of the user, wherein the digital twin is created based on a set of user specific data, a health condition of the user, a change in the health condition of the user, a shopping cycle, a monetary budget for the shopping cycle, one or more activities of the user that may occur during the shopping cycle, and a set of weather data. In an embodiment, shopping support program 122 contains digital twin component 122-A. Digital twin component 122-A of shopping support program 122 operates to perform a simulation of the digital twin to create a food buying plan for the shopping cycle. In the depicted embodiment, shopping support program 122 is a standalone program. In another embodiment, shopping support program 122 may be integrated into another software product, such as a grocery store inventory management software. In the depicted embodiment, shopping support program 122 resides on server 120. In another embodiment, shopping support program 122 may reside on user computing devices 130₁₋ _{N} or on another computing device (not shown), provided that shopping support program 122 has access to network 110. The operational steps of shopping support program 122 are depicted and described in further detail with respect to FIG. 5.

In an embodiment, users of user computing devices 130_{1-N} register with shopping support program 122 on server 120. For example, a user completes a registration process (e.g., user validation), provides information to create a user profile, and authorizes the collection, analysis, and distribution (i.e., opts-in) of relevant data on identified computing devices (e.g., on user computing devices 130_{1-N}) by shopping support program 122 on server 120. Relevant data includes, but is not limited to, personal information or data provided by the user or inadvertently provided by the user’s device without the user’s knowledge; tagged and/or recorded location information of the user (e.g., to infer context (i.e., time, place, and usage) of a location or existence); time stamped temporal information (e.g., to infer contextual reference points); and specifications pertaining to the software or hardware of the user’s device. In an embodiment, the user opts-in or opts-out of certain categories of data collection. For example, the user can opt-in to provide all requested information, a subset of requested information, or no information. In one example scenario, the user opts-in to provide time-based information, but opts-out of providing location-based information (on all or a subset of computing devices associated with the user). In an embodiment, the user opts-in or opts-out of certain categories of data analysis. In an embodiment, the user opts-in or opts-out of certain categories of data distribution. Such preferences can be stored in database 124.

Database 124 operates as a repository for data received, used, and/or generated by shopping support program 122. A database is an organized collection of data. Data includes, but is not limited to, information about user preferences (e.g., general user system settings such as alert notifications for user computing devices 130_{1-N}); information about alert notification preferences; feedback received from the user; and any other data received, used, and/or generated by shopping support program 122. Database 124 can be implemented with any type of device capable of storing data and configuration files that can be accessed and utilized by server 120, such as a hard disk drive, a database server, or a flash memory. In an embodiment, database 124 is accessed by shopping support program 122 to store and/or to access the data. In the depicted embodiment, database 124 resides on server 120. In another embodiment, database 124 may reside on another computing device, server, cloud server, or spread across multiple devices elsewhere (not shown) within distributed data processing environment 100, provided that shopping support program 122 has access to database 124.

The present invention may contain various accessible data sources, such as database 124, that may include personal and/or confidential company data, content, or information the user wishes not to be processed. Processing refers to any operation, automated or unautomated, or set of operations such as collecting, recording, organizing, structuring, storing, adapting, altering, retrieving, consulting, using, disclosing by transmission, dissemination, or otherwise making available, combining, restricting, erasing, or destroying personal and/or confidential company data. Shopping support program 122 enables the authorized and secure processing of personal data.

Shopping support program 122 provides informed consent, with notice of the collection of personal and/or confidential data, allowing the user to opt-in or opt-out of processing personal and/or confidential data. Consent can take several forms. Opt-in consent can impose on the user to take an affirmative action before personal and/or confidential data is processed. Alternatively, opt-out consent can impose on the user to take an affirmative action to prevent the processing of personal and/or confidential data before personal and/or confidential data is processed. Shopping support program 122 provides information regarding personal and/or confidential data and the nature (e.g., type, scope, purpose, duration, etc.) of the processing. Shopping support program 122 provides the user with copies of stored personal and/or confidential company data. Shopping support program 122 allows the correction or completion of incorrect or incomplete personal and/or confidential data. Shopping support program 122 allows for the immediate deletion of personal and/or confidential data.

User computing devices 130_{1-N} operate to each run user interfaces 132_{1-N}, respectively, through which a user can interact with shopping support program 122 on server 120 and to store data in and/or send data from local databases 134_{1-N}. As used herein, N represents a positive integer, and accordingly the number of scenarios implemented in a given embodiment of the present invention is not limited to those depicted in FIG. 1. In an embodiment, user computing devices 130_{1-N} are each a device that performs programmable instructions. For example, user computing devices 130_{1-N} may each be an electronic device, such as a laptop computer, a tablet computer, a netbook computer, a personal computer, a desktop computer, a smart phone, or any programmable electronic device capable of running respective user interfaces 132_{1-N} and of communicating (i.e., sending and receiving data) with shopping support program 122 via network 110. In general, user computing devices 130_{1-N} represent any programmable electronic device or a combination of programmable electronic devices capable of executing machine readable program instructions and communicating with other computing devices (not shown) within distributed data processing environment 100 via network 110. In some embodiments, user computing devices 130_{1-N} may include more than one user computing device, such as a wearable computing device. A wearable computing device, also referred to as a “wearable,” is a category of smart electronic devices that can detect, analyze, and transmit information concerning the wearer’s body (e.g., vital signs and ambient data) and, in some cases, allow immediate biofeedback to the wearer. A wearable computing device can be worn as an accessory, embedded in a clothing item, implanted in a body, or even tattooed on the skin. A wearable computing device is a hands-free device with practical uses, powered by microprocessors and enhanced with the ability to send and receive data via the internet. In the depicted embodiment, user computing devices 130_{1-N} each include a respective instance of user interfaces 132_{1-N} and local databases 134_{1-N}.

User interfaces 132_{1-N} each operate as a local user interface between shopping support program 122 on server 120 and a user of user computing devices 130_{1-N}. In some embodiments, user interfaces 132_{1-N} each are a graphical user interface (GUI), a web user interface (WUI), and/or a voice user interface (VUI) that can display (i.e., visually) or present (i.e., audibly) text, documents, web browser windows, user options, application interfaces, and instructions for operations sent from shopping support program 122 to a user via network 110. User interfaces 132_{1-N} can also display or present alerts including information (such as graphics, text, and/or sound) sent from shopping support program 122 to a user via network 110. In an embodiment, user interfaces 132_{1-N} are capable of sending and receiving data (i.e., to and from shopping support program 122 via network 110, respectively). Through user interfaces 132_{1-N}, a user can opt-in to shopping support program 122; register a user computing device; grant permission to shopping support program 122 to gather sensor data on the body movement of the user during a defined period of time through the user computing device; create a user profile; set user preferences and alert notification preferences; input user specific data; define a shopping cycle; identify one or more activities scheduled to occur during the shopping cycle; define a monetary budget for the shopping cycle; input a list of food items available in the user’s stockpile at home; input an accounting of the food items available in the user’s stockpile at home; input associated details about the food items available in the user’s stockpile at home (e.g., nutritional parameters of the food items, expiration date of the food items, etc.); receive a notification that the monetary budget for the shopping cycle is insufficient; increase the monetary budget for the shopping cycle; and receive a shopping list for the shopping cycle.

A user preference is a setting that can be customized for a particular user. A set of default user preferences are assigned to each user of shopping support program 122. A user preference editor can be used to update values to change the default user preferences. User preferences that can be customized include, but are not limited to, general user system settings, specific user profile settings, alert notification settings, and machine-learned data collection/storage settings. Machine-learned data is a user’s personalized corpus of data. Machine-learned data includes, but is not limited to, past results of iterations of shopping support program 122 including, but not limited to, data regarding one or more food items the user consumes, the frequency of the user’s consumption of the one or more food items, one or more activities the user participates in, the frequency of the user’s participation in the one or more activities, an shopping list previously generated, how strictly the user followed the shopping list, whether an amount of food on the shopping list for the shopping cycle was sufficient, whether a type of food on the shopping list for the shopping cycle was correct, and whether the monetary budget for the shopping cycle was sufficient.

Local databases 134_{1-N} operate as a repository for an unmodified digital twin representative of the user, an unmodified digital twin representative of a household of users, and user specific data. Local databases 134_{1-N} can be implemented with any type of device capable of storing data and configuration files that can be accessed and utilized by server 120, such as a hard disk drive, a database server, or a flash memory. In an embodiment, local databases 134_{1-N} are each accessed by shopping support program 122 to store and/or to access the data. In the depicted embodiment, local databases 134_{1-N} reside on respective user computing devices 130_{1-N}. In another embodiment, local databases 134_{1-N} may reside on another computing device, server, cloud server, or spread across multiple devices elsewhere (not shown) within distributed data processing environment 100, provided that shopping support program 122 has access to local databases 134_{1-N}.

Database 140 operates as a repository for a list, an accounting, and an explanation of associated details (e.g., nutritional parameters, of the food items and expiration date of the food items) of the food items available in the stockpile of a plurality of brick-and-mortar businesses, a plurality of online stores, a plurality of warehouses, and/or a plurality of supply chains. In the depicted embodiment, database 140 receives data from brick-and-mortar businesses 142_{1-N}, online businesses 144_{1-N}, warehouses 146_{1-N}, and supply chains 148_{1-N}. A brick-and-mortar business is a traditional business that has a physical store or stores where a customer can browse and purchase items in person. An online business is a business conducted entirely on the internet. The customer can browse and purchase items over the internet and have the purchased items shipped to the customer. A warehouse is a building used by manufacturers, importers, exporters, wholesalers, and transportation businesses for storing goods. A supply chain is a network between a business and its suppliers to produce and distribute a specific product to the customer. The network between the business and its suppliers includes different activities, people, entities, information, and resources. Database 140 can be implemented with any type of device capable of storing data and configuration files that can be accessed and utilized by server 120, such as a hard disk drive, a database server, or a flash memory. In an embodiment, database 140 is accessed by shopping support program 122 to store and/or to access the data. In the depicted embodiment, database 140 is a standalone database.

FIG. 2 is a block diagram illustrating the components of computing device 200, suitable for server 120 running shopping support program 122 within distributed data processing environment 100 of FIG. 1, in accordance with an embodiment of the present invention. It should be appreciated that FIG. 2 provides only an illustration of one implementation and does not imply any limitations with regard to the environments in which different embodiments can be implemented. Many modifications to the depicted environment can be made.

As depicted, computing device 200 includes communications fabric 202, processor(s) 204, memory 206, persistent storage 208, communications unit 210, input/output (I/O) interface(s) 212, and cache 216. Communications fabric 202 provides communications between memory 206, cache 216, persistent storage 208, input/output (I/O) interface(s) 212, and communications unit 210. Communications fabric 202 can be implemented with any architecture designed for passing data and/or control information between processors (such as microprocessors, communications and network processors, etc.), system memory, peripheral devices, and any other hardware components within a system. For example, communications fabric 202 can be implemented with one or more buses or a cross switch.

Memory 206 and persistent storage 208 are computer readable storage media. In this embodiment, memory 206 includes random access memory (RAM). In general, memory 206 can include any suitable volatile or non-volatile computer readable storage media. Cache 216 is a fast memory that enhances the performance of computer processor(s) 204 by holding recently accessed data, and data near accessed data, from memory 206.

Program instructions and data (e.g., software and data) used to practice embodiments of the present invention may be stored in persistent storage 208 and in memory 206 for execution by one or more of the respective processor(s) 204 via cache 216. In an embodiment, persistent storage 208 includes a magnetic hard disk drive. Alternatively, or in addition to a magnetic hard disk drive, persistent storage 208 can include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage 208 may also be removable. For example, a removable hard drive may be used for persistent storage 208. Other examples include optical and magnetic disks, thumb drives, and smart cards that are inserted into a drive for transfer onto another computer readable storage medium that is also part of persistent storage 208. Software and data can be stored in persistent storage 208 for access and/or execution by one or more of the respective processor(s) 204 via cache 216. With respect to user computing device 130_{1-N}, software and data includes user interface 132_{1-N}. With respect to server 120, software and data includes shopping support program 122.

Communications unit 210, in these examples, provides for communications with other data processing systems or devices. In these examples, communications unit 210 includes one or more network interface cards. Communications unit 210 may provide communications through the use of either or both physical and wireless communications links. Program instructions and data (e.g., software and data) used to practice embodiments of the present invention may be downloaded to persistent storage 208 through communications unit 210.

I/O interface(s) 212 allows for input and output of data with other devices that may be connected to each computer system. For example, I/O interface(s) 212 may provide a connection to external device(s) 218, such as a keyboard, a keypad, a touch screen, and/or some other suitable input device. External device(s) 218 can also include portable computer readable storage media, such as, for example, thumb drives, portable optical or magnetic disks, and memory cards. Program instructions and data (e.g., software and data) used to practice embodiments of the present invention can be stored on such portable computer readable storage media and can be loaded onto persistent storage 208 via I/O interface(s) 212. I/O interface(s) 212 also connect to display 220.

Display 220 provides a mechanism to display data to a user and may be, for example, a computer monitor.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:
On-demand self-service: a cloud consumer can unilaterally provision computing capabilities, such as server time and network storage, as needed automatically without requiring human interaction with the service’s provider.
Broad network access: capabilities are available over a network and accessed through standard mechanisms that promote use by heterogeneous thin or thick client platforms (e.g., mobile phones, laptops, and PDAs).
Resource pooling: the provider’s computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).
Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.
Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:
Software as a Service (SaaS): the capability provided to the consumer is to use the provider’s applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.
Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.
Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:
Private cloud: the cloud infrastructure is operated solely for an organization. It may be managed by the organization or a third party and may exist on-premises or off-premises.
Community cloud: the cloud infrastructure is shared by several organizations and supports a specific community that has shared concerns (e.g., mission, security requirements, policy, and compliance considerations). It may be managed by the organizations or a third party and may exist on-premises or off-premises.
Public cloud: the cloud infrastructure is made available to the general public or a large industry group and is owned by an organization selling cloud services.
Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

FIG. 3 is a block diagram illustrating an exemplary cloud computing environment, generally designated cloud computing environment 300, in accordance with an embodiment of the present invention. As shown, cloud computing environment 300 includes one or more cloud computing nodes 310 with which local computing devices used by cloud consumers, such as, for example, personal digital assistant (PDA) or cellular telephone 320A, desktop computer 320B, laptop computer 320C, and/or automobile computer system 320N may communicate. Nodes 310 may communicate with one another. They may be grouped (not shown) physically or virtually, in one or more networks, such as Private, Community, Public, or Hybrid clouds as described hereinabove, or a combination thereof. This allows cloud computing environment 300 to offer infrastructure, platforms, and/or software as services for which a cloud consumer does not need to maintain resources on a local computing device. It is understood that the types of computing devices 320A-N shown in FIG. 3 are intended to be illustrative only and that computing nodes 310 and cloud computing environment 300 can communicate with any type of computerized device over any type of network and/or network addressable connection (e.g., using a web browser).

FIG. 4 is a block diagram illustrating a set of functional abstraction model layers, generally designated 400, of the exemplary cloud computing environment (e.g., cloud computing environment 300), in accordance with an embodiment of the present invention. It should be understood in advance that the components, layers, and functions shown in FIG. 4 are intended to be illustrative only and embodiments of the invention are not limited thereto. As depicted, the following layers and corresponding functions are provided:

Hardware and software layer 460 includes hardware and software components. Examples of hardware components include: mainframes 461; RISC (Reduced Instruction Set Computer) architecture based servers 462; servers 463; blade servers 464; storage devices 465; and networks and networking components 466. In some embodiments, software components include network application server software 467 and database software 468.

Virtualization layer 470 provides an abstraction layer from which the following examples of virtual entities may be provided: virtual servers 471; virtual storage 472; virtual networks 473, including virtual private networks; virtual applications and operating systems 474; and virtual clients 475.

In one example, management layer 480 may provide the functions described below. Resource provisioning 481 provides dynamic procurement of computing resources and other resources that are utilized to perform tasks within the cloud computing environment. Metering and Pricing 482 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 483 provides access to the cloud computing environment for consumers and system administrators. Service level management 484 provides cloud computing resource allocation and management such that required service levels are met. Service Level Agreement (SLA) planning and fulfillment 485 provides pre-arrangement for, and procurement of, cloud computing resources for which a future requirement is anticipated in accordance with an SLA.

Workloads layer 490 provides examples of functionality for which the cloud computing environment may be utilized. Examples of workloads and functions which may be provided from this layer include: mapping and navigation 491; software development and lifecycle management 492; virtual classroom education delivery 493; data analytics processing 494; transaction processing 495; and, in the context of the illustrated embodiments of the present invention, various optimization workloads and functions 496.

FIG. 5 is a flowchart, generally designated 500, illustrating the operational steps of shopping support program 122, on server 120 within distributed data processing environment 100 of FIG. 1, in accordance with an embodiment of the present invention. In an embodiment, shopping support program 122 operates to create a digital twin representative of a user and the food consumption of the user. In an embodiment, shopping support program 122 operates to perform a simulation of the digital twin to create a food buying plan for a shopping cycle. It should be appreciated that the process depicted in FIG. 5 illustrates one possible iteration of the process flow, which may be repeated each time a food buying plan is generated for a user.

In step 505, shopping support program 122 (hereinafter referred to as “program 122”) creates a digital twin representative of a user in an unmodified state. In another embodiment, program 122 creates a digital twin representative of a household of users in an unmodified state. A digital twin in an unmodified state is a template of a generic digital twin. In an embodiment, program 122 stores the digital twin in a database, i.e., in a personal cloud, e.g., in local database 134_{1-N}.

In step 510, program 122 mines for data associated with the user from a plurality of sources to personalize the digital twin. In an embodiment, responsive to creating the digital twin representative of the user in the unmodified state, program 122 mines for data associated with the user from a plurality of sources to personalize the digital twin.

In an embodiment, program 122 collects user specific data from the user profile stored in the database, i.e., in the personal cloud, e.g., in local database 134_{1-N}. In an embodiment, program 122 collects user specific data from the user. In an embodiment, program 122 enables the user to input user specific data through user interface 132_{1-N} of user computing device 130_{1-N}. User specific data may include, but is not limited to, biometric identifying data and health data. Biometric identifying data includes body measurements and calculations related to human characteristics of the user. Biometric identifying data is distinctive, measurable characteristics of the user. Biometric identifying data is categorized as physiological characteristics (e.g., fingerprints, palm prints, palm veins, hand geometry, face recognition, iris recognition, retina, DNA) and as behavioral characteristics (i.e., related to a pattern of behavior of a user, e.g., behavioral profiling, voice, signature, gait). Health data is any data related to the diagnosis, care, and treatment of a health condition, a reproductive outcome, and quality of life for the user. This includes clinical metrics, environmental information, socioeconomic information, and behavioral information pertinent to the health and wellness of the user. Health data is typically collected and used when the user interacts with a health care system. More specifically, health data may include, but is not limited to, data found in an electronic health record (e.g., the user’s name, date of birth, general numerical information (i.e., vital signs such as heart rate, respiratory rate, and temperature), diagnostic-related information (i.e., laboratory test results from blood tests, genetic tests, culture tests, and x-rays), and treatment information (i.e., prescriptions)).

In an embodiment, program 122 collects weather data from a weather Application Programming Interface (API). A weather API provides access to current and forecasted weather conditions, seasonal and sub-seasonal forecasts, lifestyle indices, and severe weather and historical weather data.

In an embodiment, program 122 extracts information from the user specific data. The information extracted from the user specific data relates to the health condition of the user and the changes in the health condition of the user based on food intake patterns and based on weather patterns. In an embodiment, program 122 simulates the health condition of the user. For example, program 122 accommodates user A’s nut allergy. In another example, program 122 accommodates user B’s need to eat a low sodium diet. In yet another example, program 122 accommodates user C’s need to eat a low-fat diet.

In step 515, program 122 enables the user to define the shopping cycle. In an embodiment, responsive to mining for data associated with the user from the plurality of sources to personalize the digital twin, program 122 enables the user to define the shopping cycle, i.e., the future period of time for which the user is shopping. In an embodiment, program 122 enables the user to define the shopping cycle through user interface 132_{1-N} of user computing device 130_{1-N}. The shopping cycle may be, for example, the next day, the next 7 days, the next 14 days, or the next 30 days.

In an embodiment, program 122 identifies one or more activities of the user that may occur during the shopping cycle based on historical patterns. In another embodiment, program 122 identifies one or more activities of the user that may occur during the shopping cycle based on the biometric identifying data of the user. In another embodiment, program 122 identifies one or more activities of the user that may occur during the shopping cycle based on the information extracted from the user specific data relating to the current health condition of the user and the changes in the health condition of the user). In another embodiment, program 122 identifies one or more activities of the user that may occur during the shopping cycle based on information extracted from a calendar of the user on user computing device 130_{1-N}. In another embodiment, program 122 enables the user to identify one or more activities scheduled to occur during the shopping cycle through user interface 132_{1-N} of user computing device 130_{1-N}.

In an embodiment, program 122 enables the user to define a monetary budget for the shopping cycle. In an embodiment, program 122 enables the user to define a monetary budget for the shopping cycle through user interface 132_{1-N} of user computing device 130_{1-N}.

In step 520, program 122 personalizes the digital twin. In an embodiment, responsive to enabling the user to define the shopping cycle, program 122 personalizes the digital twin. In an embodiment, program 122 personalizes (i.e., updates or modifies) the digital twin to include user specific data (e.g., biometric identifying data and health data), the current health condition of the user and the changes in the health condition of the user, the shopping cycle, the one or more activities of the user during the shopping cycle, the monetary budget for the shopping cycle, and weather data. In an embodiment, program 122 personalizes the digital twin to be representative of the various types of information collected and therewith combined to make a food buying plan personalized to the user. In an embodiment, program 122 stores the updated digital twin in the database, e.g., local database 134_{1-N}.

In step 525, program 122 performs a simulation of the digital twin to create a food buying plan for the shopping cycle. In an embodiment, responsive to personalizing the digital twin, program 122 performs a simulation of the digital twin to create a food buying plan for the shopping cycle.

In an embodiment, program 122 collects a list of food items available in the user’s stockpile at home from the user. In an embodiment, program 122 enables the user to input the list of food items available in the user’s stockpile at home through user interface 132_{1-N} of user computing device 130_{1-N}. In an embodiment, program 122 collects an accounting of the food items available in the user’s stockpile at home from the user (i.e., a quantity of the food items available). In an embodiment, program 122 enables the user to input the accounting of the food items available in the user’s stockpile at home through user interface 132_{1-N} of user computing device 130_{1-N}. In an embodiment, program 122 collects associated details about the food items available in the user’s stockpile at home (e.g., nutritional parameters of the food items, expiration date of the food items) from the user. In an embodiment, program 122 enables the user to input the associated details through user interface 132_{1-N} of user computing device 130_{1-N}. In another embodiment, program 122 collects the associated details about the food items available in the user’s stockpile at home from a database, e.g., database 126. The nutritional parameter of a food item is an indication of the contribution of the food item to the nutrient content of the user’s diet. The nutritional parameter of the food item depends on the quantity of a food items digested and absorbed and the amount of essential nutrients contained in the food items (i.e., protein, fat, carbohydrate, minerals, vitamins). This value can be affected by soil and growing conditions, handling and storage, and processing. In an embodiment, program 122 identifies one or more recipes that can be created using the food items available in the user’s stockpile at home. In an embodiment, program 122 identifies how the one or more recipes that can be created using the food items available in the user’s stockpile at home would impact the health condition of the user.

In an embodiment, program 122 considers what food items are available in the stockpile of brick-and-mortar businesses 142_{1-N}, online businesses 144_{1-N}, warehouses 146_{1-N}, and supply chains 148_{1-N}. In an embodiment, program 122 collects a list of food items available in the stockpile of brick-and-mortar businesses 142_{1-N}, online businesses 144_{1-N}, warehouses 146_{1-N}, and supply chains 148_{1-N} from a database, e.g., database 140. In an embodiment, program 122 collects an accounting of the food items available in the stockpile of brick-and-mortar businesses 142_{1-N}, online businesses 144_{1-N}, warehouses 146_{1-N}, and supply chains 148_{1-N} from the database, e.g., database 140. In an embodiment, program 122 collects associated details (e.g., nutritional parameters of the food items, expiration date of the food items, etc.) about the food items available in the stockpile of brick-and-mortar businesses 142_{1-N}, online businesses 144_{1-N}, warehouses 146_{1-N}, and supply chains 148_{1-N} from the database, e.g., database 140.

In step 530, program 122 creates the food buying plan for the shopping cycle. In an embodiment, responsive to performing the simulation of the digital twin to create the food buying plan for the shopping cycle, program 122 creates the food buying plan for the shopping cycle. In an embodiment, program 122 creates the food buying plan for the shopping cycle considering user specific data (i.e., biometric identifying data and health data), the current health condition of the user and the changes in the health condition of the user, the shopping cycle, the one or more activities of the user during the shopping cycle, the monetary budget for the shopping cycle, and weather data. In an embodiment, program 122 creates the food buying plan for the shopping cycle considering what food items are available in the user’s stockpile at home and what food items are available in the stockpile of brick-and-mortar businesses 142_{1-N}, online businesses 144_{1-N}, warehouses 146_{1-N}, and supply chains 148_{1-N}.

In an embodiment, program 122 runs health simulations while the food buying plan for the shopping cycle is created. In an embodiment, program 122 runs health simulations based on the food items on the food buying plan for the shopping cycle. In an embodiment, program 122 runs health simulations to identify how the food items on the food buying plan for the shopping cycle will impact the health condition of the user.

In step 535, program 122 identifies the price of each food items on the food buying plan for the shopping cycle at brick-and-mortar businesses 142_{1-N} and at online businesses 144_{1-N}. In an embodiment, responsive to creating the food buying plan for the shopping cycle, program 122 identifies the price of each food items on the food buying plan for the shopping cycle at brick-and-mortar businesses 142_{1-N} and at online businesses 144_{1-N}. In an embodiment, program 122 identifies available offers on each food item on the food buying plan for the shopping cycle at brick-and-mortar businesses 142_{1-N} and at online businesses 144_{1-N}.

In step 540, program 122 creates a shopping list for the user. In an embodiment, responsive to identifying the price of each food items on the food buying plan for the shopping cycle at brick-and-mortar businesses 142_{1-N} and at online businesses 144_{1-N}, program 122 creates a shopping list for the user. In another embodiment, program 122 creates a shopping list for the household of users.

In decision 545, program 122 determines whether the monetary budget for the shopping cycle is sufficient to purchase the food items on the shopping list. In an embodiment, responsive to creating the shopping listing for the user, program 122 determines whether the monetary budget for the shopping cycle is sufficient to purchase the food items on the shopping list. If program 122 determines the monetary budget for the shopping cycle is sufficient to purchase the food items on the shopping list (decision 545, YES branch), then program 122 proceeds to step 555, outputting the shopping list for the shopping cycle to the user. If program 122 determines the monetary budget for the shopping cycle is insufficient to purchase the food items on the shopping list (decision 545, NO branch), then program 122 proceeds to step 550, notifying the user that the monetary budget for the shopping cycle is insufficient.

In step 550, program 122 outputs a notification to the user. In an embodiment, responsive to determining the monetary budget for the shopping cycle is insufficient to purchase the food items on the shopping list, program 122 outputs a notification to the user. In an embodiment, program 122 outputs a notification to the user through user interface 132_{1-N} of user computing device 130_{1-N}. In an embodiment, program 122 outputs a notification to the user informing the user that the monetary budget for the shopping cycle is insufficient. In an embodiment, program 122 enables the user to change the monetary budget for the shopping cycle. In an embodiment, program 122 enables the user to increase the monetary budget for the shopping cycle through user interface 132_{1-N} of user computing device 130_{1-N}. In another embodiment, responsive to the user choosing not to change the monetary budget for the shopping cycle, program 122 modifies the food items on the shopping list to fit the monetary budget for the shopping cycle.

In step 555, program 122 outputs the shopping list for the shopping cycle to the user. In an embodiment, responsive to determining the monetary budget for the shopping cycle is sufficient to purchase the food items on the shopping list, program 122 outputs the shopping list for the shopping cycle to the user. In an embodiment, program 122 outputs the shopping list for the shopping cycle to the user through user interface 132_{1-N} of user computing device 130_{1-N}.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

While particular embodiments of the present invention have been shown and described here, it will be understood to those skilled in the art that, based upon the teachings herein, changes and modifications may be made without departing from the embodiments and its broader aspects. Therefore, the appended claims are to encompass within their scope all such changes and modifications as are within the true spirit and scope of the embodiments. Furthermore, it is to be understood that the embodiments are solely defined by the appended claims. It will be understood by those with skill in the art that if a specific number of an introduced claim element is intended, such intent will be explicitly recited in the claim, and in the absence of such recitation no such limitation is present. For a non-limiting example, as an aid to understand, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to embodiments containing only one such element, even when the same claim includes the introductory phrases “at least one” or “one or more” and indefinite articles such as “a” or “an”, the same holds true for the use in the claims of definite articles.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart illustrations and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart illustrations and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart illustrations and/or block diagram block or blocks.

The flowchart illustrations and block diagrams in the Figures illustrate the architecture, functionality, and operation of possible implementations of systems, methods, and computer program products according to various embodiments of the present invention. In this regard, each block in the flowchart illustrations or block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each flowchart illustration and/or block of the block diagrams, and combinations of flowchart illustration and/or blocks in the block diagrams, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method comprising:
creating, by one or more processors, a digital twin representative of a user in an unmodified state;
mining, by the one or more processors, for data associated with the user from a plurality of sources to personalize the digital twin;
performing, by the one or more processors, a simulation of the digital twin to create a food buying plan for a shopping cycle;
identifying, by the one or more processors, a price of each food item on the food buying plan and one or more available offers on each food item on the food buying plan to determine whether a monetary budget for the shopping cycle is sufficient; and
responsive to determining the monetary budget for the shopping cycle is sufficient, outputting, by the one or more processors, a shopping list for the shopping cycle based on the simulation of the digital twin.

2. The computer-implemented method of claim 1, wherein mining for the data associated with the user from the plurality of sources to personalize the digital twin further comprises:
collecting, by the one or more processors, a set of user specific data from the user and from a user profile of the user, wherein the set of user specific data includes biometric identifying data and health data;
collecting, by the one or more processors, a set of weather data from a weather application programming interface; and
extracting, by the one or more processors, information from the set of user specific data relating to a health condition of the user.

3. The computer-implemented method of claim 2, further comprising:
prior to performing the simulation of the digital twin to create the food buying plan for the shopping cycle, enabling, by the one or more processors, the user to define the shopping cycle;
responsive to receiving a defined shopping cycle from the user, identifying, by the one or more processors, one or more activities of the user that may occur during the shopping cycle based on one or more historical patterns, a set of biometric identifying data of the user, information extracted from the set of user specific data relating to the health condition of the user, and a calendar of the user; and
receiving, by the one or more processors, the monetary budget for the shopping cycle.

4. The computer implemented method of claim 3, further comprising:
updating, by the one or more processors, the digital twin to include the set of user specific data, the set of weather data, the health condition of the user, the shopping cycle, the one or more activities of the user that may occur during the shopping cycle, and the monetary budget for the shopping cycle.

5. The computer-implemented method of claim 1, wherein performing the simulation of the digital twin to create the food buying plan for the shopping cycle further comprises:
collecting, by the one or more processors, a list of food items available in a stockpile of the user, an accounting of the food items available in the stockpile of the user, and one or more associated details of the food items available in the stockpile of the user;
identifying, by the one or more processors, one or more recipes that can be created using the food items available in the stockpile of the user; and
collecting, by the one or more processors, a list of food items available in a stockpile of a plurality of brick-and-mortar businesses, a plurality of online stores, a plurality of warehouses, and a plurality of supply chains; an accounting of the food items available in the stockpile of the plurality of brick-and-mortar businesses, the plurality of online stores, the plurality of warehouses, and the plurality of supply chains; and one or more associated details about the food items available in the stockpile of the plurality of brick-and-mortar businesses, the plurality of online stores, the plurality of warehouses, and the plurality of supply chains.

6. The computer-implemented method of claim 5, further comprising:
creating, by the one or more processors, the food buying plan for the shopping cycle; and
running, by the one or more processors, a health simulation on the digital twin to determine how the food items on the food buying plan for the shopping cycle will impact the health condition of the user.

7. The computer-implemented method of claim 1, wherein identifying the price of each food item on the food buying plan and the one or more available offers on each food item on the food buying plan to determine whether the monetary budget for the shopping cycle is sufficient further comprises:
responsive to determining the monetary budget for the shopping cycle is insufficient to purchase the food items on the food buying plan for the shopping cycle, notifying, by the one or more processors, the user that the monetary budget for the shopping cycle is insufficient and that the user must increase the monetary budget for the shopping cycle; and
enabling, by the one or more processors, the user to change the monetary budget for the shopping cycle.

8. A computer program product comprising:
one or more computer readable storage media and program instructions stored on the one or more computer readable storage media, the program instructions comprising:
program instructions to create a digital twin representative of a user in an unmodified state;
program instructions to mine for data associated with the user from a plurality of sources to personalize the digital twin;
program instructions to perform a simulation of the digital twin to create a food buying plan for a shopping cycle;
program instructions to identify a price of each food item on the food buying plan and one or more available offers on each food item on the food buying plan to determine whether a monetary budget for the shopping cycle is sufficient; and
responsive to determining the monetary budget for the shopping cycle is sufficient, program instructions to output a shopping list for the shopping cycle based on the simulation of the digital twin.

9. The computer program product of claim 8, wherein mining for the data associated with the user from the plurality of sources to personalize the digital twin further comprises:
program instructions to collect a set of user specific data from the user and from a user profile of the user, wherein the set of user specific data includes biometric identifying data and health data;
program instructions to collect a set of weather data from a weather application programming interface; and
program instructions to extract information from the set of user specific data relating to a health condition of the user.

10. The computer program product of claim 9, further comprising:
prior to performing the simulation of the digital twin to create the food buying plan for the shopping cycle, program instructions to enable the user to define the shopping cycle;
responsive to receiving a defined shopping cycle from the user, program instructions to identify one or more activities of the user that may occur during the shopping cycle based on one or more historical patterns, a set of biometric identifying data of the user, information extracted from the set of user specific data relating to the health condition of the user, and a calendar of the user; and
program instructions to receive the monetary budget for the shopping cycle.

11. The computer program product of claim 10, further comprising:
program instructions to update the digital twin to include the set of user specific data, the set of weather data, the health condition of the user, the shopping cycle, the one or more activities of the user that may occur during the shopping cycle, and the monetary budget for the shopping cycle.

12. The computer program product of claim 8, wherein performing the simulation of the digital twin to create the food buying plan for the shopping cycle further comprises:
program instructions to collect a list of food items available in a stockpile of the user, an accounting of the food items available in the stockpile of the user, and one or more associated details of the food items available in the stockpile of the user;
program instructions to identify one or more recipes that can be created using the food items available in the stockpile of the user; and
program instructions to collect a list of food items available in a stockpile of a plurality of brick-and-mortar businesses, a plurality of online stores, a plurality of warehouses, and a plurality of supply chains; an accounting of the food items available in the stockpile of the plurality of brick-and-mortar businesses, the plurality of online stores, the plurality of warehouses, and the plurality of supply chains; and one or more associated details about the food items available in the stockpile of the plurality of brick-and-mortar businesses, the plurality of online stores, the plurality of warehouses, and the plurality of supply chains.

13. The computer program product of claim 12, further comprising:
program instructions to create the food buying plan for the shopping cycle; and
program instructions to run a health simulation on the digital twin to determine how the food items on the food buying plan for the shopping cycle will impact the health condition of the user.

14. The computer program product of claim 8, wherein identifying the price of each food item on the food buying plan and the one or more available offers on each food item on the food buying plan to determine whether the monetary budget for the shopping cycle is sufficient further comprises:
responsive to determining the monetary budget for the shopping cycle is insufficient to purchase the food items on the food buying plan for the shopping cycle, program instructions to notify the user that the monetary budget for the shopping cycle is insufficient and that the user must increase the monetary budget for the shopping cycle; and
program instructions to enable the user to change the monetary budget for the shopping cycle.

15. A computer system comprising:
one or more computer processors;
one or more computer readable storage media;
program instructions collectively stored on the one or more computer readable storage media for execution by at least one of the one or more computer processors, the stored program instructions comprising:
program instructions to create a digital twin representative of a user in an unmodified state;
program instructions to mine for data associated with the user from a plurality of sources to personalize the digital twin;
program instructions to perform a simulation of the digital twin to create a food buying plan for a shopping cycle;
program instructions to identify a price of each food item on the food buying plan and one or more available offers on each food item on the food buying plan to determine whether a monetary budget for the shopping cycle is sufficient; and
responsive to determining the monetary budget for the shopping cycle is sufficient, program instructions to output a shopping list for the shopping cycle based on the simulation of the digital twin.

16. The computer system of claim 15, wherein mining for the data associated with the user from the plurality of sources to personalize the digital twin further comprises:
program instructions to collect a set of user specific data from the user and from a user profile of the user, wherein the set of user specific data includes biometric identifying data and health data;
program instructions to collect a set of weather data from a weather application programming interface; and
program instructions to extract information from the set of user specific data relating to a health condition of the user.

17. The computer system of claim 16, further comprising:
prior to performing the simulation of the digital twin to create the food buying plan for the shopping cycle, program instructions to enable the user to define the shopping cycle;
responsive to receiving a defined shopping cycle from the user, program instructions to identify one or more activities of the user that may occur during the shopping cycle based on one or more historical patterns, a set of biometric identifying data of the user, information extracted from the set of user specific data relating to the health condition of the user, and a calendar of the user; and
program instructions to receive the monetary budget for the shopping cycle.

18. The computer system of claim 15, wherein performing the simulation of the digital twin to create the food buying plan for the shopping cycle further comprises:
program instructions to collect a list of food items available in a stockpile of the user, an accounting of the food items available in the stockpile of the user, and one or more associated details of the food items available in the stockpile of the user;
program instructions to identify one or more recipes that can be created using the food items available in the stockpile of the user; and
program instructions to collect a list of food items available in a stockpile of a plurality of brick-and-mortar businesses, a plurality of online stores, a plurality of warehouses, and a plurality of supply chains; an accounting of the food items available in the stockpile of the plurality of brick-and-mortar businesses, the plurality of online stores, the plurality of warehouses, and the plurality of supply chains; and one or more associated details about the food items available in the stockpile of the plurality of brick-and-mortar businesses, the plurality of online stores, the plurality of warehouses, and the plurality of supply chains.

19. The computer system of claim 18, further comprising:
program instructions to create the food buying plan for the shopping cycle; and
program instructions to run a health simulation on the digital twin to determine how the food items on the food buying plan for the shopping cycle will impact the health condition of the user.

20. The computer system of claim 15, wherein identifying the price of each food item on the food buying plan and the one or more available offers on each food item on the food buying plan to determine whether the monetary budget for the shopping cycle is sufficient further comprises:
responsive to determining the monetary budget for the shopping cycle is insufficient to purchase the food items on the food buying plan for the shopping cycle, program instructions to notify the user that the monetary budget for the shopping cycle is insufficient and that the user must increase the monetary budget for the shopping cycle; and
program instructions to enable the user to change the monetary budget for the shopping cycle.
